(19) United States
(12) Patent Application Publication
(10) Pub. No.: US 2024/0115135 A1
(43) Pub. Date: Apr. 11, 2024

(54) NEAR-INFRARED IMAGING OPTODE

(71) Applicant: Cortirio Limted, Sedgefield Durham (GB)

(72) Inventors: Patrick Beldon, Sedgefield Durham (GB); Paul Macey, Sedgefield Durham (GB)

(73) Assignee: Cortirio Limted, Sedgefield Durham (GB)

(21) Appl. No.: 18/275,762

(22) PCT Filed: Feb. 3, 2022
(86) PCT No.: PCT/GB2022/050282

§ 371 Date: Aug. 3, 2023

(30) Foreign Application Priority Data
Feb. 4, 2021 (GB) 2101566.4

(51) Int. Cl. A61B 5/00; G06T 7/00; H04N 23/21; H04N 25/20

(52) U.S. Cl. CPC A61B 5/0042; A61B 5/0073; A61B 5/0075; G06T 7/0012; H04N 23/21; H04N 25/20; A61B 2562/0238; A61B 2562/164; G02B 6/06; G06T 2207/10048; G06T 2207/10072; G06T 2207/30016; G06T 2207/30104; G06T 2207/30196

(57) ABSTRACT

Disclosed is a near-infrared imaging optode (200) and associated system and method for transmitting near-infrared radiation into and/or receiving near-infrared radiation from a subjects head. The optode (200) comprises a plurality of resilient optical fibres (201) arranged to transmit and/or receive corresponding near-infrared radiation. The plurality of optical fibres (201) each comprise a distal end (203) arranged to make contact with a subjects head. The distal ends (203) of the plurality of optical fibres (201) are movable relative to each other.

### TECHNICAL FIELD

The invention relates to near-infrared (NIR) imaging optodes, systems and methods.

### BACKGROUND

Near-infrared imaging techniques such as near-infrared spectroscopy (N IRS) or tomography can be used to identify features within an object.

Near-infrared imaging techniques can be applied to neuroimaging to identify structural or functional features within a subject's head. In such applications, near-infrared radiation is transmitted into a subject's head and corresponding output radiation, which has propagated through the subject's head, is detected and analysed.

Information about the interior of the subject's head can be inferred from changes the NIR radiation undergoes as it propagates through the subject's head based, in particular, on the fact that NIR radiation is absorbed differently by oxygenated haemoglobin and deoxygenated haemoglobin.

Whilst the spatial resolution that can be achieved using certain NIRS based techniques is low compared to other imaging techniques such as computed tomography (CT), they are nevertheless considered promising techniques for rapidly identifying features indicative of pathologies such as intra-cranial hematomas.

In a typical example of N IRS, a headset comprising an array of optodes is placed on a subject's head. Each optode is typically a single rigid fibre optic element which is connected at one end in the headset to either a NIR transmitter or NIR receiver and is configured at the other end to contact the surface of the subject's head for either directing NIR radiation into the subject's head or for receiving NIR radiation that has propagated through the subject's head.

Output NIR radiation is detected and analysed to determine changes that the NIR radiation has undergone as it propagated through the subject's head. Corresponding parameters (for example signal attenuation and phase-shift) are input to a computing system running specially adapted data analysis software.

This data analysis software implements a mathematical, computational or machine learning model of the propagation path of the NIR radiation. By inputting the parameters from the detected output NIR radiation, characteristics of the region of the subject's head through which the radiation has propagated can be estimated. These characteristics can then be analysed to determine, for example, changes in blood oxygenation within the cortex indicative of activity levels within different regions of the brain, or whether the characteristics include features indicative of a target pathology such as an intra-cranial hematoma.

Such mathematical, computational or machine learning models are typically based on assumptions that the optodes have “ideal” contact with the surface of the subject's head. For example, it is assumed that optodes engage with the subject's head with a high level of physical contact between the distal ends of the optodes and the subject's head.

However, when using conventional optodes, ideal contact between the distal ends of the optodes and the surface of the subject's head may not be consistently possible due to various factors that can reduce or entirely prevent physical contact between the distal ends of the optodes and the subject's head. Such factors can include variations in the subject's head shape, incorrect optode positioning relative to the subject's head and/or hair or other structures getting trapped between the distal ends of the optodes and the surface of the subject's head.

As a result of poor contact between the optodes and the subject's head, the accuracy with which characteristics of the region of the subject's head can be estimated is reduced.

It is an object of certain embodiments of the invention to address one or more of the above described disadvantages.

It is an object of certain embodiments of the invention to provide a near-infrared imaging optode that can provide improved contact with the surface of a subject's head to thereby improve the accuracy with which it is possible to perform near-infrared neuroimaging.

### SUMMARY OF THE INVENTION

In accordance with a first aspect of the invention there is provided a near-infrared imaging optode for transmitting near-infrared radiation into and/or receiving near-infrared radiation from a subject's head. The optode comprises: a plurality of resilient optical fibres arranged to transmit and/or receive corresponding near-infrared radiation, the plurality of optical fibres each comprising a distal end arranged to make contact with a subject's head, wherein the distal ends of the plurality of optical fibres are movable relative to each other, and wherein the optode is arranged to be coupled with a near-infrared radiation detector unit which comprises an imaging sensor.

Optionally, the plurality of optical fibres are arranged to deform on contact with a subject's head and to return to an initial shape after removal from contact with the subject's head.

Optionally, the near-infrared imaging optode further comprises a support sleeve enclosing the plurality of optical fibres along a portion of their length and arranged such that the distal ends of the plurality of optical fibres extend out from the support sleeve to make contact with a subject's head.

Optionally, the near-infrared imaging optode further comprises a substrate and wherein a proximal end of each of the plurality of optical fibres is secured to or integral with the substrate.

Optionally, each of the plurality of optical fibres comprises an enclosed optically transmissive core.

Optionally, the optically transmissive core is composed of silica or plastic.

Optionally, each of the plurality of optical fibres has a diameter of between approximately 0.2 mm and 1 mm.

Optionally, each of the plurality of optical fibres has a length of between 5 mm and 15 mm.

Optionally, each of the plurality of optical fibres comprises a substantially flat distal end.

Optionally, the substantially flat distal end comprises a bevelled edge.

Optionally, each of the plurality of optical fibres has a substantially convex distal end.

Optionally, each of the plurality of optical fibres has a substantially concave distal end.

Optionally, the imaging sensor comprises a plurality of pixels.

Optionally, each optical fibre of the optode is arranged to be coupled to at least one pixel of the imaging sensor.

Optionally, each of the plurality of pixels of the imaging sensor is arranged to image all wavelengths of near-infrared radiation received by the optode.

Optionally, the optode is a near-infrared spectroscopy or near-infrared tomography optode.

Optionally, the optode is not a spectrophotometry optode.

In accordance with a second aspect of the invention there is provided a near-infrared imaging system for identifying a target feature in a subject's head, the system comprising: an optode array comprising a plurality of optodes arranged to transmit near-infrared radiation into a region of a subject's head and to detect corresponding near-infrared radiation emitted from the region of the subject's head, at least one optode of the optode array comprising a plurality of resilient optical fibres arranged to transmit and/or receive corresponding near-infrared radiation, the plurality of optical fibres each comprising a distal end arranged to make contact with a subject's head, wherein the distal ends of the plurality of optical fibres are movable relative to each other, and wherein the at least one optode is arranged to be coupled with a near-infrared radiation detector unit which comprises an imaging sensor. The system further comprising: a data processing system configured to process detected near-infrared radiation in accordance with a near-infrared imaging model to identify the presence or absence of a target feature within the subject's head.

Optionally, the system further comprises a plurality of near-infrared radiation detector units each coupled to a respective optode of the optode array.

Optionally, each of the plurality of near-infrared radiation detector units comprises an imaging sensor for detecting near-infrared radiation detected by a respective optode that is coupled to the near-infrared radiation detector unit.

Optionally, the imaging sensor comprises a plurality of pixels.

Optionally, at least one pixel of the imaging sensor is coupled to each optical fibre of the plurality of optical fibres of a respective optode.

Optionally, each of the plurality of pixels of the imaging sensor is arranged to image all wavelengths of near-infrared radiation received by the optode.

Optionally, the imaging sensor is a charge coupled device (CCD) or complementary metal oxide semiconductor (CMOS) imaging sensor.

Optionally, the target feature is a change in perfusion or liquid content of biological tissue.

Optionally, the target feature is indicative of a target pathology.

Optionally, the target pathology is an intracranial haematoma, intracranial haemorrhage, or change in blood flow, blood oxygenation or blood volume characteristic of cerebral ischaemia.

Optionally, the near-infrared imaging system is a near-infrared spectroscopy or near-infrared tomography system.

Optionally, the near-infrared imaging system is not a spectrophotometry system.

Optionally, the data processing system is configured to perform a demodulation process on detected near-infrared radiation prior to processing the detected near-infrared radiation in accordance with the near-infrared imaging model.

In accordance with a third aspect of the invention there is provided a method of transmitting near-infrared radiation into and/or receiving near-infrared radiation from a subject's head, the method comprising: bringing a near-infrared imaging optode into contact with a subject's head, the optode comprising a plurality of resilient optical fibres arranged to transmit and/or receive corresponding near-infrared radiation, the plurality of optical fibres each comprising a distal end arranged to make contact with the subject's head, wherein the distal ends of the optical fibres are movable relative to each other; and transmitting and/or receiving near-infrared radiation via one or more optical fibres of the plurality of optical fibres that are in contact with the subject's head.

Optionally, the method further comprises: removing the near-infrared imaging optode from contact with the subject's head such that the plurality of optical fibres return to an initial shape.

In accordance with embodiments of the invention, there is provided a near-infrared imaging device comprising a near-infrared optode coupled to a near-infrared radiation detector unit. The optode comprises a plurality of resilient optical fibres arranged to transmit and/or receive corresponding near-infrared radiation. The plurality of optical fibres each comprise a distal end arranged to make contact with a subject's head. The distal ends of the plurality of optical fibres are movable relative to each other. The near-infrared radiation detector unit comprises an imaging sensor for detecting near-infrared radiation detected by the optode. In certain embodiments, the imaging sensor comprises a plurality of pixels.

Advantageously, aspects of the invention provide a near-infrared optode and associated system and method that can provide improved contact with the surface of a subject's head. In particular, the optode can provide a larger surface area of contact between the distal end of the optode and the surface of a subject's head, and can do so in a more reliable manner than conventional optodes. This means that coupling of NIR radiation between the subject's head and the optode is improved. This can improve the accuracy with which a region of the subject's head can be characterised, for example to identify a target pathology such as an intracranial hematoma.

The optode comprises a “bundle” of optical fibres. The optical fibres are mechanically resilient and arranged so that at least at their respective distal ends they are free to move relative to each other. This allows the optical fibres to individually bend (also referred to herein as to “deform”) on contact with the surface of a subject's head i.e. during the action of engaging the optode with the subject's head, the individual optical fibres can be forced from a substantially straight initial condition into a bent condition. For example, the optical fibres can individually bend along their length and/or the distal ends of the optical fibres can be displaced relative to each other (e.g. splayed in or out) as they make contact with the surface of a subject's head.

This enables the individual optical fibres of the optode to bend on contact with a subject's head in a way that can increase the surface area of contact between the optode and the surface of the subject's head. Advantageously, in this way the optode can provide an improved level of physical contact with the subject's head in situations that may, for conventional optodes comprising a single rigid fibre optic element, limit or entirely prevent contact between the optode and the subject's head, such as when an optode is non-perpendicularly positioned relative to the head or when hair or other structures are trapped between an optode and the surface of the subject's head.

Advantageously, in accordance with certain embodiments of the invention, a plurality of optodes can be provided to form an optode array. In such embodiments, each optode (comprising a “bundle” of optical fibres) can be connected to a separate near-infrared radiation detector unit. Each near-infrared radiation detector unit can include an imaging sensor comprising a plurality of pixels. Advantageously, in such an arrangement the imaging sensor associated with each optode can detect near-infrared radiation received via each optical fibre of the bundle of optical fibres.

Advantageously, using an optode comprising a plurality of individual optical fibres in combination with an imaging sensor comprising a plurality of pixels can improve the accuracy and resolution of imaging performed by the system. This is because NIR signals received via each optical fibre of an optode can be individually measured and processed.

This can further improve the accuracy with which it is possible to perform near-infrared neuroimaging on a subject by increasing the resolution with which near-infrared signals can be detected. Further, signal processing techniques can be performed on NIR signals received from individual optical fibres of an optode, for example to account for differences in the quality of optical connection between individual optical fibres and the subject's head.

Typically, the optode is coupled with the near-infrared radiation detector unit such that received near-infrared radiation passes directly from the optode to the near-infrared radiation detector unit without passing through another structure. This is in contrast with other systems such as spectrophotometry systems which include one or more optical components positioned between the optodes and detectors, such optical components used to split received near-infrared radiation into different wavelengths prior to imaging. Advantageously, directly coupling the optode with the near-infrared radiation detector unit allows the system to be made more compact and portable compared with existing systems such as spectrophotometry systems.

Various further features and aspects of the invention are defined in the claims.

### BRIEF DESCRIPTION OF THE DRAWINGS

Embodiments of the present invention will now be described by way of example only with reference to the accompanying drawings where like parts are provided with corresponding reference numerals and in which:

FIG. 1 is a simplified schematic diagram showing a near-infrared spectroscopy system in accordance with an embodiment of the invention;

FIG. 2 is a simplified schematic diagram showing an optode in accordance with an embodiment of the invention;

FIG. 3a is a simplified schematic diagram showing a cross-sectional view of an optode in accordance with certain embodiments of the invention prior to contact with a subject's head;

FIG. 3b is a simplified schematic diagram showing a cross-sectional view of the optode shown in FIG. 3a in contact with a subject's head;

FIG. 4 is a simplified schematic diagram showing an optode in accordance with a further embodiment of the invention;

FIG. 5 is a simplified schematic diagram showing an optode array in accordance with a further embodiment of the invention;

FIG. 6 is a simplified schematic diagram showing a near-infrared imaging system in accordance with a further embodiment of the invention;

FIG. 7 is a simplified schematic diagram showing one of the NIR radiation detector units of the system of FIG. 6 coupled with an optode of a receive optode array;

FIG. 8 provides a simplified schematic diagram showing a plan view of an optode connected to an imaging sensor of a NIR radiation detector unit in accordance with certain embodiments of the invention; and

FIGS. 9a-9d provide simplified schematic diagrams showing the distal ends of optical fibres in accordance with certain embodiments of the invention.

### DETAILED DESCRIPTION

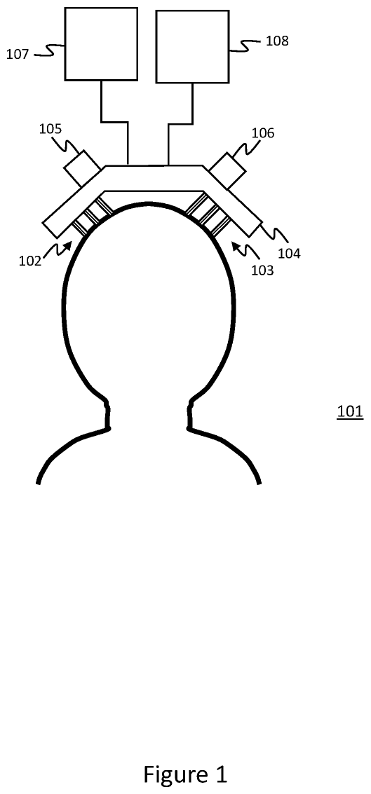

FIG. 1 is a simplified schematic diagram showing a near-infrared (NIR) imaging system in accordance with an embodiment of the invention. The near-infrared imaging system is for identifying a target feature in an object. In this embodiment, the object is a subject's head, and the target feature is a feature indicative of the presence of a target pathology within the subject's head. Examples of target pathologies include an intracranial haematoma, intracranial haemorrhage, or change in blood flow, blood oxygenation or blood volume characteristic of intracranial ischaemia.

In this embodiment, the near-infrared imaging system is a near-infrared spectroscopy (NIRS) system, such as a structural or functional NIRS (fNIRS) system.

In keeping with conventional systems, the NIR system 101 comprises an optode array comprising a plurality of transmit optodes 102 and a plurality of receive optodes 103 coupled to NIR radiation transmit/receive unit 104 (provided, for example in a headset). The transmit/receive unit 104 comprises a NIR radiation generator unit 105 coupled to the transmit optodes 102 and a NIR radiation detector unit 106 coupled to the receive optodes 103. In use, the optode array is positioned on a subject's head.

NIR radiation is transmitted into the subject's head via the plurality of transmit optodes 102 and corresponding NIR radiation that has propagated through the subject's head is received by the plurality of receive optodes 103. Operation of the NIR radiation transmit/receive unit 104 and in particular the NIR radiation generator unit 105 and NIR radiation detector unit 106 is controlled by a control unit 107, typically provided by a suitably programmed computing device including a memory and processor.

The transmit/receive unit 104 comprises componentry configured to generate output signals conveying data relating to the NIR radiation received by the receive optodes 103 and detected by the NIR radiation detector unit 106. These output signals are communicated via a suitable signal line to a data processing system 108, typically provided by a suitably programmed computing device.

The data processing system 108 is configured to analyse this data using a near-infrared spectroscopy imaging model to generate output data relating to the region of the subject's head through which the NIR radiation has propagated, for example, output data indicating whether or not a target pathology is present.

The imaging model is a mathematical model of the propagation path through the region of the subject's head which is configured to generate estimates of characteristics of the propagation path based on changes that a known signal has undergone as it has propagated through the region of the subject's head.

These characteristics can then be used to determine, for example, whether or not a target pathology is present in the propagation path.

More specifically, the imaging model is configured to compare differences between the NIR radiation generated by the NIR radiation generator unit 105 and the NIR radiation detected by the NIR radiation detector unit 106 to determine changes that the NIR radiation has undergone as it propagates through the region of the subject's head.

These changes (typically signal attenuation, phase shifts and frequency shifts) are then quantized and input to the model to generate output characteristics corresponding to estimates of the characteristics of the propagation path—i.e. characteristics of the region of the subject's head through which the NIR radiation has propagated.

These output characteristics can be input to a diagnostic function (also provided by software running on the data processing system 108) which is configured to determine if they are indicative of the presence of a target pathology.

In certain embodiments, the data processing system is configured to perform a demodulation process on the data generated by the NIR radiation detector unit before the data is analysed by the near-infrared spectroscopy imaging model. The demodulation process can be used to reduce noise present in the data and thereby improve the imaging model accuracy.

In such embodiments, the near-infrared radiation signals generated by the NIR radiation generator unit are modulated in amplitude. Typically, such modulation follows a continuous frequency sine-wave profile.

The data processing system is configured to perform a demodulation process on data from the NIR radiation detector unit corresponding to the received signal. The demodulation process involves multiplying the received signal by a carrier signal having the same frequency as the modulation signal. The resulting signal is then filtered to obtain the demodulated signal.

One or more optodes of the receive optodes 103 are coupled with the NIR radiation detector unit 106 such that received near-infrared radiation passes directly from respective optodes to the NIR radiation detector unit 106 without passing through another structure. This is in contrast with other systems such as spectrophotometry systems which include one or more optical components positioned between optodes and detectors, such optical components used to split received near-infrared radiation into different wavelengths prior to imaging. In certain embodiments the NIR radiation detector unit 106 comprises an imaging sensor comprising a plurality of pixels. In such embodiments, each of the plurality of pixels of the imaging sensor can be arranged to image all wavelengths of near-infrared radiation received by an optode.

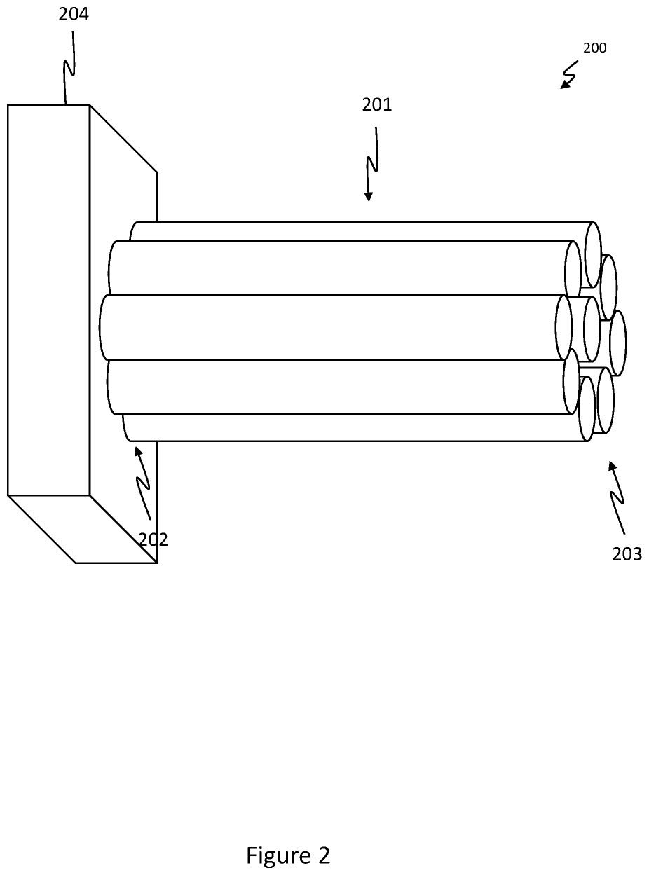

In contrast with conventional systems, one or more optodes of the optode array is configured as described in more detail below and in particular with reference to FIGS. 2-4.

FIG. 2 is a simplified schematic diagram showing a near-infrared imaging optode in accordance with an embodiment of the invention.

The optode 200 comprises a plurality of optical fibres 201. In this embodiment, the optode 200 comprises nine individual optical fibres. It will however be understood that the optode 200 could be provided with more or fewer than nine optical fibres. The plurality of optical fibres forming a single optode are also referred to herein as a bundle.

The optical fibres 201 each comprise an elongate optically transmissive core enclosed by an optically insulating outer layer. The core is composed of a suitable material such as glass (silica) fibre or plastic. The optical fibres 201 are mechanically resilient so that they deform when a force is applied to them and return to an original shape when the force is removed. The optical fibres 201 are configured so that they are deformable when subject to the typical forces experienced when an optode makes contact with a surface of a subject's head. The optical fibres 201 are arranged to transmit and/or receive near-infrared radiation along their length.

The optical fibres 201 each include a proximal end 202 that is coupled, directly or via another structure, to a NIR radiation generator and/or NIR radiation detector unit. In this embodiment, the proximal ends 202 of the optical fibres 201 are secured by a suitable adhesive to an optode substrate 204. The optical fibres 201 each include a distal end 203 that is arranged to make contact with the surface of a subject's head to transmit near-infrared radiation into and/or receive near-infrared radiation from a region of the subject's head.

The distal ends 203 of the plurality of optical fibres 201 are movable relative to each other. At least at the distal ends 203 of the optical fibres 201, the optical fibres 201 are not physically secured together. For example, there is no adhesive or sleeve provided at the distal ends 203 securing the individual optical fibres 201 together.

This together with the resilient nature of the optical fibres 201 allows the optical fibres 201 to individually change shape (also referred to herein as “deform”) on contact with the surface of a subject's head (for example the subject's scalp or other structures such as hair overlying the scalp). For example, on contact, the distal ends 203 of the optical fibres 201 can splay out or in relative to each other and/or the optical fibres 201 can bend along a portion of their length.

This can improve the surface area of contact between the optode 200 and the subject's head.

For example, if a strand of a subject's hair overlies part of an optode contact point, as the optode 200 is moved towards the subject's head, some optical fibres will make contact with the strand of hair. As the optode 200 continues to be moved towards the subject's head, the optical fibres that are in contact with the strand of hair will deform to allow other optical fibres of the optode 200 to make contact with the subject's head. In this way, an object that physically obstructs some of the optical fibres does not prevent other optical fibres from making contact. In such examples, this can result in at least part of the optode 200 (i.e. at least some of the individual optical fibres) making contact with the subject's head to enable NIR radiation to be transmitted and/or received.

In another example, if the optode 200 is misaligned, for example because it is not completely perpendicular to the subject's head, optical fibres on one side of the optode 200 (the “nearside” optodes) will make contact with the subject's head first. As the optode 200 continues to be moved towards the head, the nearside optodes will deform until the remainder of the optical fibres of the optode 200 make contact with the subject's head. In such examples, this can result in a larger surface area of contact being made between the optode 200 and the subject's head, which can improve NIR radiation transmission or reception.

Typically, the optical fibres 201 are configured to be resiliently deformable so that they return to their initial shape after being removed from contact with a subject's head. This means that the optode 200 can be reused and/or repositioned on the subject's head. During the action of engaging the optode with the subject's head, the individual optical fibres bend from a substantially straight initial condition into a bent condition. After removal from contact, the individual optical fibres return to the initial substantially straight condition.

In certain embodiments, the optode substrate 204 can form part of the optode 200.

In certain embodiments, the optode substrate 204 can have one or more further optodes secured to it to provide an optode array. In such embodiments, the optode substrate can be flexible to accommodate the shape of the subject's head.

Alternatively, in certain embodiments the optode substrate 204 is not provided and the proximal ends 202 of the optical fibres 201 are arranged to be secured and interface directly with a NIR transmitter/receiver.

It will be understood that the optical fibres can be of a range of suitable lengths and diameters. Preferably, in certain embodiments the optical fibres each have a length of between approximately 5 mm and 15 mm. Preferably, in certain embodiments, the optical fibres have a diameter of between approximately 0.2 mm and 1 mm.

However, in certain examples other suitable lengths and diameters of optical fibres can be provided. For example, in certain examples, each optical fibre can have a length of between approximately 0.05 mm and 80 mm and/or a diameter of between approximately 0.001 mm and 5 mm.

In certain embodiments, the optode 200 is not a spectrophotometry optode. In certain embodiments, the optode 200 is used in a near-infrared imaging system that is not a spectrophotometry system.

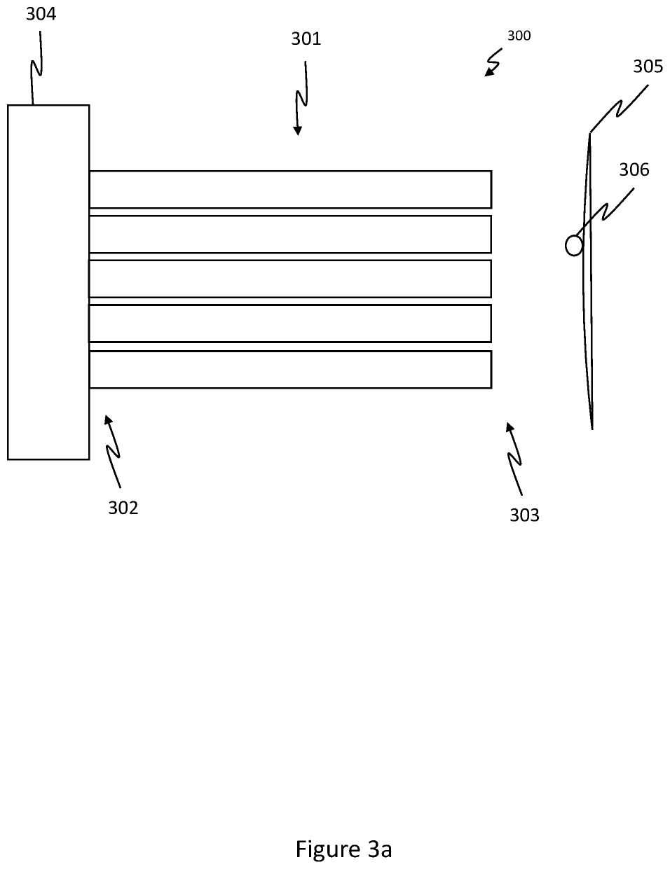

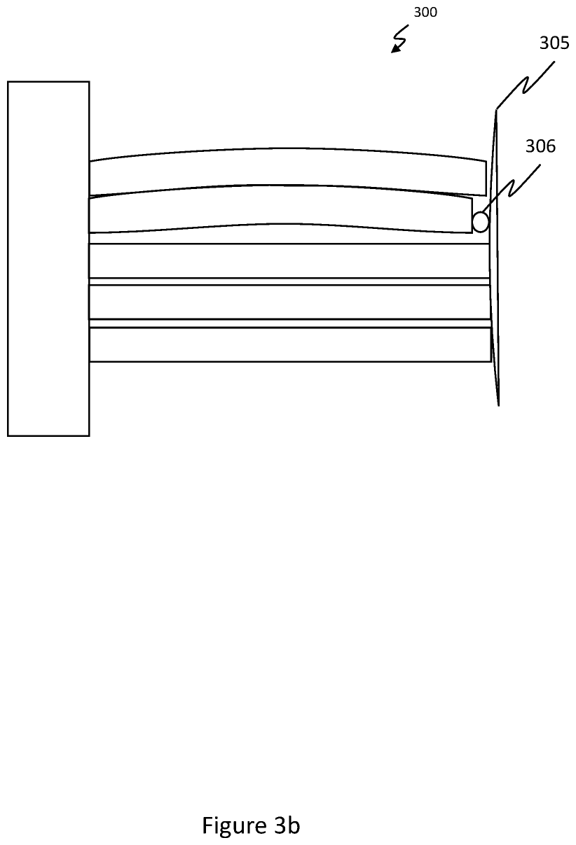

FIG. 3a is a simplified schematic diagram showing a cross-sectional view of an optode in accordance with certain embodiments of the invention prior to contact with a subject's head. FIG. 3b shows the optode of FIG. 3a in contact with the subject's head.

The optode 300 is configured and operates substantially in accordance with the optode described with reference to FIG. 2. The optode 300 includes a bundle of optical fibres 301 each having a proximal 302 and distal end 303. The optode 300 comprises a substrate 304 to which the proximal ends 302 of the optical fibres are secured.

Part of a surface of a subject's head 305 is shown in FIG. 3a. An object 306 such as a hair is shown on the surface of the subject's head 305.

As shown in FIG. 3a, prior to contact with the subject's head 305, the optode 300 is in an initial configuration in which the optical fibres 301 are substantially straight and are evenly spaced relative to each other.

In use, the optode 300 is applied to the subject's head 305. This step typically occurs when an optode array, of which the optode 300 forms a part, is placed on the subject's head prior to NIRS imaging.

The optode 300 is moved towards the subject's head 305. One or more of the optical fibres 301 first make contact with the object 306.

The optode 300 continues to be moved towards the subject's head 305 such that movement of the optode 300 towards the subject's head causes relative movement of the optical fibres such that any optical fibres in contact with the object 306 individually deform.

As shown in FIG. 3b, which shows the optode in contact with a subject's head, the optical fibre that is in contact with the object 306 has bent along its length. As shown in FIG. 3b, by virtue of the distal ends of the optical fibres being movable relative to each other, and the resilient nature of the optical fibres, the majority of the optical fibres of the optode 300 can still make contact with the subject's head 305.

It will be understood that a similar process takes place when the optode 300 is applied to the subject's head 305 when it is misaligned (e.g. is not completely perpendicular to the subject's head 305). Optical fibres on one side of the optode 300 (the “nearside” optodes) make contact with the subject's head 305 first. As the optode 300 continues to be moved towards the head 305, the nearside optodes will change shape in a manner that allows the remainder of the optical fibres of the optode 300 make contact with the subject's head 305.

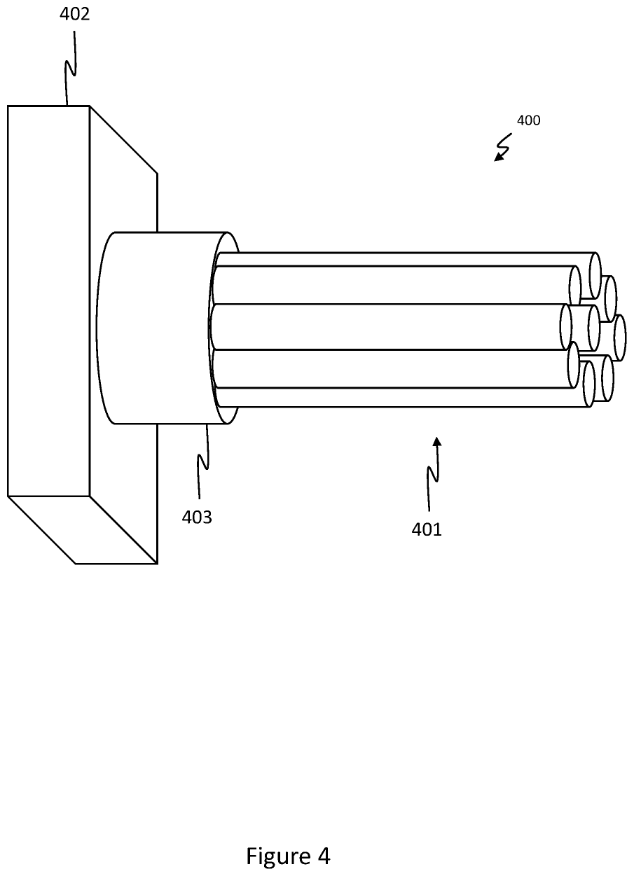

FIG. 4 is a simplified schematic diagram showing an optode in accordance with a further embodiment of the invention.

The optode 400 substantially corresponds with the optode described with reference to FIG. 2 except as otherwise described and depicted.

Similar to the embodiment of FIG. 2, the optode 400 includes a plurality of optical fibres 401 forming an optode bundle secured to a substrate 402.

In this embodiment, the optode 400 also comprises a support sleeve 403. The support sleeve 403 encloses the plurality of optical fibres 401 adjacent to the proximal ends of the optical fibres. The distal ends of the optical fibres extend out from the support sleeve 403.

In this way, the support sleeve supports the optical fibres such that the optical fibres are held together as a bundle but the distal ends of the optical fibres are able to move relative to each other.

In this embodiment the support sleeve 403 is secured to or integral with the substrate 402. It will however be understood that in other embodiments, the support sleeve 403 can be provided independent of a substrate 402.

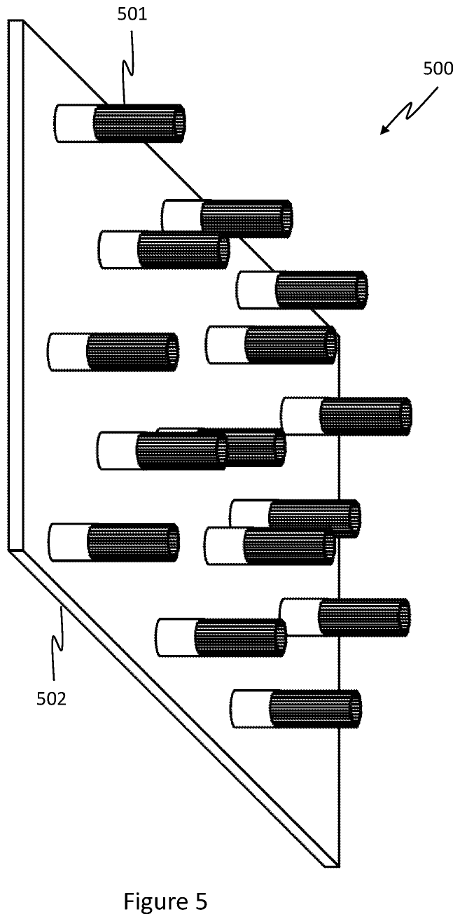

FIG. 5 is a simplified schematic diagram showing an optode array in accordance with a further embodiment of the invention.

The optode array 500 comprises a plurality of optodes 501 secured to a substrate 502. In certain embodiments, the substrate 502 is flexible to accommodate the shape of a subject's head.

In this embodiment, the plurality of optodes 501 are configured substantially in accordance with the optode described with reference to FIG. 4. It will however be understood that in certain embodiments, the optode array 500 can be made up of other types of optodes as described herein.

In use, typically the optode array 500 is a first array and a corresponding second array is provided. The optodes of one array can be used as transmit optodes and the optodes of the other array can be used as receive optodes in a NIR spectroscopy system as described herein.

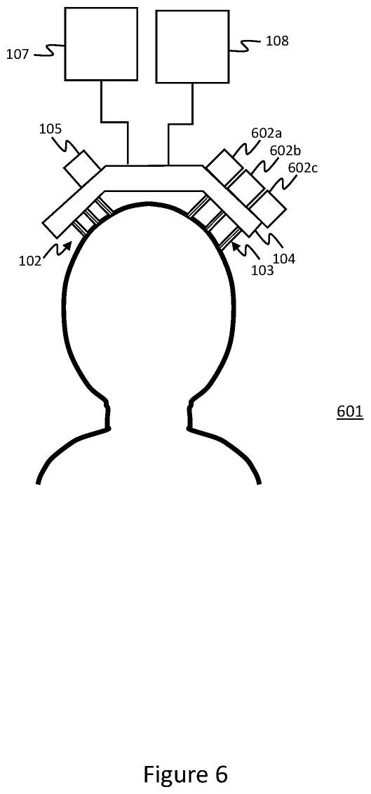

FIG. 6 is a simplified schematic diagram showing a near-infrared imaging (NIR) system in accordance with an embodiment of the invention. The NIR system 601 substantially corresponds with the system of FIG. 1 except as otherwise described and depicted.

In keeping with the system of FIG. 1, the NIR system 601 includes an optode array comprising a plurality of transmit optodes 102 and a plurality of receive optodes 103 coupled to NIR radiation transmit/receive unit 104. The transmit/receive unit 104 comprises an NIR radiation generator unit 105 coupled to the transmit optodes 102. The NIR system 601 includes a control unit 107 and a data processing system 108.

The optodes of the optode array are configured as described in more detail herein and in particular with reference to FIGS. 2-4.

In contrast with the system of FIG. 1, which includes a single NIR radiation detector unit coupled to the plurality of receive optodes 103, in this embodiment the transmit/receive unit 104 comprises a plurality of NIR radiation detector units 602a 602b 602c. Each of the receive optodes 103 is coupled to one of the NIR radiation detector units of the plurality of NIR radiation detector units 602a 602b 602c.

Each NIR radiation detector unit 602a 602b 602c comprises a suitable imaging sensor such as a CCD (charge coupled device) or CMOS (complementary metal oxide semiconductor) imaging sensor for detecting NIR radiation. Each imaging sensor includes multiple pixels. For example, in certain embodiments, the imaging sensors can be made up of a 20×20 array of pixels. In this way, each of the receive optodes 103, which are each composed of a bundle of resilient optical fibres, is coupled to its own NIR radiation detector unit and associated imaging sensor.

Typically, each optical fibre is coupled to at least one pixel of the imaging sensor. In certain embodiments, each optical fibre is coupled to a predetermined group of pixels of the imaging sensor.

Advantageously, this can enable signal processing to be performed on individual optical fibres of an optode. This can further improve the resolution of imaging performed by the system. Additionally, signal processing techniques can be performed on signals received at individual optical fibres to account for differences in the quality of optical connection between individual optical fibres of an optode and the subject's head. For example, where some fibres of an optode bundle are not in contact with a subject's head, such fibres can be identified based on the signals received.

However, in alternative embodiments the imaging sensor comprises a single detector. It will be understood that in such embodiments any suitable detector can be used, such as a photodiode, phototransistor or single photon avalanche diode (SPAD).

The transmit/receive unit 104 comprises componentry configured to generate output signals conveying data relating to the NIR radiation received by the receive optodes 103 and detected by the NIR radiation detector units 602a 602b 602c. These output signals are communicated via a suitable signal line to the data processing system 108, typically provided by a suitably programmed computing device, for analysis.

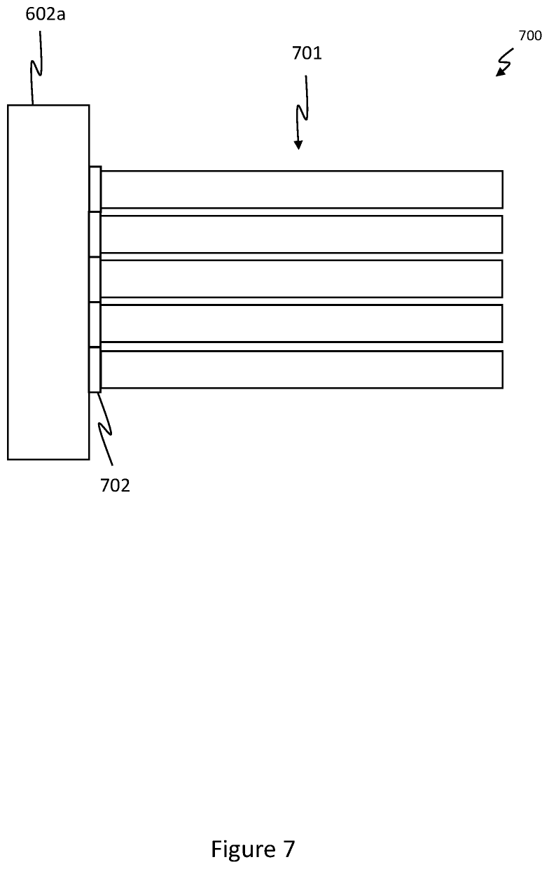

FIG. 7 is a simplified schematic diagram showing one of the NIR radiation detector units 602a of the system of FIG. 6 coupled with an optode 700 of the receive array. The optode 700 comprises a plurality of resilient optical fibres 701.

The NIR radiation detector unit 602a is associated with a single receive optode 700. The NIR radiation detector unit 602a comprises an imaging sensor 702. The imaging sensor 702 comprises a plurality of pixels. Each pixel of the imaging sensor 702 is coupled to a respective optical fibre. The imaging sensor 702 can thereby detect NIR radiation received via each optical fibre of the plurality of optical fibres 701. This can improve the accuracy with which it is possible to perform near-infrared neuroimaging on a subject.

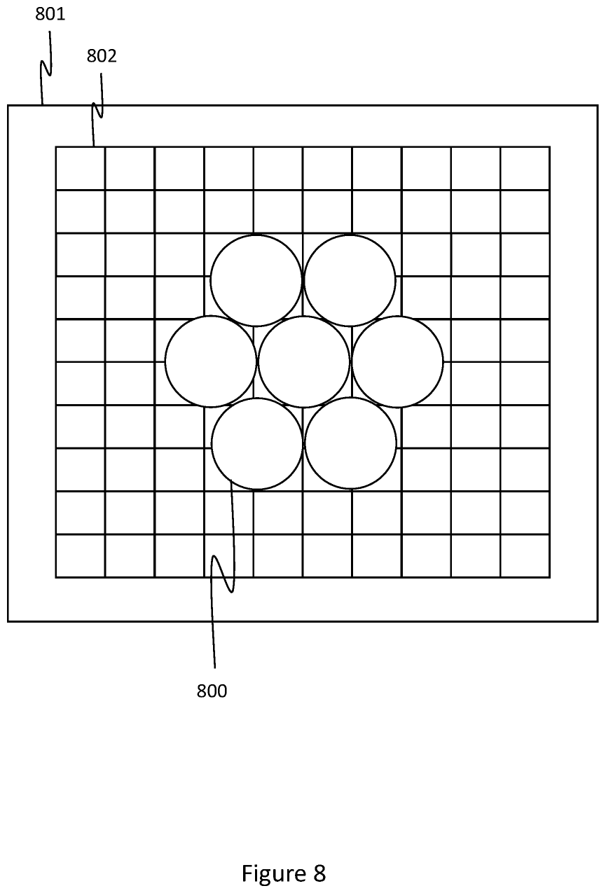

FIG. 8 provides a simplified schematic diagram showing a plan view of an optode connected to an imaging sensor of a NIR radiation detector unit in accordance with certain embodiments of the invention. The optode and NIR radiation detector unit substantially correspond with the optode and NIR radiation detector unit described with reference to FIG. 7 except as otherwise described and depicted.

The optode comprises a plurality of resilient optical fibres 800. The NIR radiation detector unit 801 comprises an imaging sensor. The imaging sensor comprises a plurality of pixels 802.

The proximal ends of the optical fibres 800 of the optode are coupled with the pixels of the imaging sensor such that NIR radiation that travels down each of the optical fibres can be detected by the pixels of the imaging sensor.

In this embodiment, each optical fibre of the optode is optically coupled with more than one pixel of the imaging sensor.

In the embodiment shown in FIG. 8, the optode comprises seven optical fibres and each of the optical fibres is coupled with four pixels of the imaging sensor. It will be understood that in other embodiments, different arrangements can be provided. For example the optode can include more or fewer than seven optical fibres and each optical fibre can be coupled with more or fewer than four pixels of an imaging sensor.

Advantageously, using an optode comprising a plurality of individual optical fibres in combination with an imaging sensor comprising a plurality of pixels can improve the accuracy and resolution of imaging performed by the system.

This is because NIR signals received via each optical fibre of an optode can be individually measured and processed.

The ability to measure and process NIR signals received via each individual optical fibre can increase the imaging resolution of the system because the spaced apart optical fibres of an optode allow NIR radiation to be measured at more points on a subject's head. The higher resolution imaging data from the optodes can be fed into the near-infrared imaging model to improve the accuracy of the output of the model.

Additionally, the ability to measure and process NIR signals received via each individual optical fibre can further improve the accuracy by allowing image processing or filtering steps to be performed on data from the optodes of a system. For example, as described herein in some circumstances some of the optical fibres of an optode may have less than an optimal amount of contact, or no contact at all, with the surface of a subject's head.

For example, when a hair on the subject's head blocks contact of one of the optical fibres, this optical fibre will not pass any NIR signals back to its associated pixels of the imaging sensor. In accordance with embodiments of the invention, any optical fibres that have a sub-optimal level of contact can be individually identified, for example, using a data processing step performed by the data processing device. Signals received via optical fibres that have a sub-optimal level of contact can then be excluded from input to the near-infrared imaging model. This can further improve the accuracy of the output of the model because only high quality NIR data is input to the model.

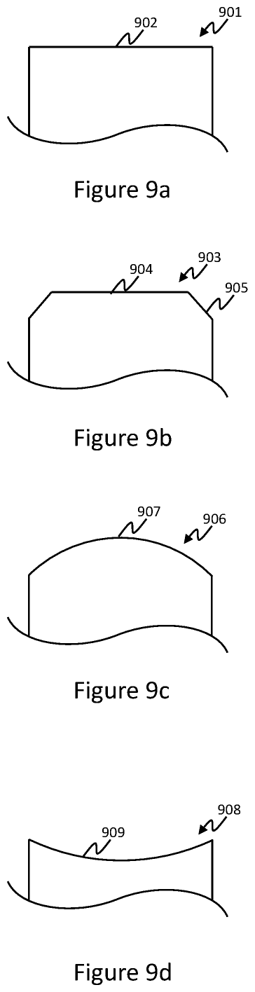

FIGS. 9a-9d provide simplified schematic diagrams showing optical fibres in accordance with certain embodiments of the invention. The optical fibres of FIGS. 9a-9d have different shaped distal ends. The optical fibres of FIGS. 9a-9d substantially correspond with those described herein except as otherwise described and depicted. In certain embodiments, the optodes described herein can use optical fibres of the type shown in FIGS. 9a-9d.

FIG. 9a shows an optical fibre that has a substantially flat distal end 901. The distal end 901 includes a substantially planar surface 902 that extends across the whole distal end 901 of the optical fibre.

Advantageously, optical fibres of the type shown in FIG. 9a can maximise the surface area of contact between the optical fibre and the subject's head. This can improve the NIR coupling between the optical fibre and the subject's head.

FIG. 9b shows an optical fibre that has a substantially flat distal end 903 with a bevelled edge. The distal end 903 includes a substantially planar surface 904 that extends across part of the distal end 903. The bevelled edge 905 provides a surface that meets with the edge of the planar surface 904 at a non-perpendicular angle.

Advantageously, an optode made up of optical fibres of the type shown in FIG. 9b can be more comfortable to wear while maintaining a high level of NIR coupling between the optical fibre and the subject's head because the bevelled edge 905 reduces areas of high pressure on the subject's head at the edges of each optical fibre while the substantially planar surface 904 maintains a large surface area of contact with the subject's head.

FIG. 9c shows an optical fibre that has a substantially convex distal end 906. The distal end 906 includes a substantially dome shaped surface 907 that extends across the distal end 906.

Advantageously, an optode made up of optical fibres of the type shown in FIG. 9c can be more comfortable to wear because the shape of the distal end 906 reduces areas of high pressure on the subject's head caused by each optical fibre. Additionally, optical fibres of the type shown in FIG. 9c have improved optical transmission properties because the shape of the distal end 906 provides a lensing effect that focusses transmitted NIR light into a smaller cone angle on the head. Optical fibres of the type shown in FIG. 9c can be particularly advantageous when used as part of a transmit optode.

FIG. 9d shows an optical fibre that has a substantially concave distal end 908. The distal end 908 includes a surface 909 extending across the distal end 908 that curves inwardly towards the proximal end of the optical fibre.

Advantageously, optical fibres of the type shown in FIG. 9d have improved optical transmission properties because the shape of the distal end 908 provides a lensing effect that allows collection of NIR light from a wider cone angle. Optical fibres of the type shown in FIG. 9d can be particularly advantageous when used as part of a receive optode.

Embodiments of the invention have been described in the context of near-infrared spectroscopy. It will be understood, however, that techniques disclosed herein can be applied, with suitable modification, to other near-infrared imaging techniques such as near-infrared tomography.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A near-infrared imaging optode for transmitting near-infrared radiation into and/or receiving near-infrared radiation from a subject's head, the optode comprising:
a plurality of resilient optical fibres arranged to transmit and/or receive corresponding near-infrared radiation, the plurality of optical fibres each comprising a distal end arranged to make contact with a subject's head, wherein the distal ends of the plurality of optical fibres are movable relative to each other, and wherein the optode is arranged to be coupled with a near-infrared radiation detector unit which comprises an imaging sensor.

2. A near-infrared imaging optode as claimed in claim 1, wherein the plurality of optical fibres are arranged to deform on contact with a subject's head and to return to an initial shape after removal from contact with the subject's head.

3. A near-infrared imaging optode as claimed in claim 1, further comprising a support sleeve enclosing the plurality of optical fibres along a portion of their length and arranged such that the distal ends of the plurality of optical fibres extend out from the support sleeve to make contact with a subject's head.

4. A near-infrared imaging optode as claimed in claim 1, further comprising a substrate and wherein a proximal end of each of the plurality of optical fibres is secured to or integral with the substrate.

5. A near-infrared imaging optode as claimed in claim 1, wherein each of the plurality of optical fibres comprises an enclosed optically transmissive core.

6. A near-infrared imaging optode as claimed in claim 5, wherein the optically transmissive core is composed of silica or plastic.

7. A near-infrared imaging optode as claimed in claim 1, wherein each of the plurality of optical fibres has a diameter of between approximately 0.2 mm and 1 mm.

8. A near-infrared imaging optode as claimed in claim 1, wherein each of the plurality of optical fibres has a length of between 5 mm and 15 mm.

9. A near-infrared imaging optode as claimed in claim 1, wherein each of the plurality of optical fibres comprises a substantially flat distal end.

10. A near-infrared imaging optode as claimed in claim 9, wherein the substantially flat distal end comprises a bevelled edge.

11. A near-infrared imaging optode as claimed in claim 1, wherein each of the plurality of optical fibres has a substantially convex distal end.

12. A near-infrared imaging optode as claimed in claim 1, wherein each of the plurality of optical fibres has a substantially concave distal end.

13. A near-infrared imaging optode as claimed in claim 1, wherein the imaging sensor comprises a plurality of pixels.

14. A near-infrared imaging optode as claimed in claim 13, wherein each optical fibre of the optode is arranged to be coupled to at least one pixel of the imaging sensor.

15. A near-infrared imaging optode as claimed in claim 1, wherein the optode is a near-infrared spectroscopy or near-infrared tomography optode.

16. A near-infrared imaging system for identifying a target feature in a subject's head, the system comprising:
an optode array comprising a plurality of optodes arranged to transmit near-infrared radiation into a region of a subject's head and to detect corresponding near-infrared radiation emitted from the region of the subject's head, at least one optode of the optode array comprising a plurality of resilient optical fibres arranged to transmit and/or receive corresponding near-infrared radiation, the plurality of optical fibres each comprising a distal end arranged to make contact with a subject's head, wherein the distal ends of the plurality of optical fibres are movable relative to each other, and wherein the at least one optode is arranged to be coupled with a near-infrared radiation detector unit which comprises an imaging sensor; and
a data processing system configured to process detected near-infrared radiation in accordance with a near-infrared imaging model to identify the presence or absence of a target feature within the subject's head.

17. A near-infrared imaging system according to claim 16, further comprising a plurality of near-infrared radiation detector units each coupled to a respective optode of the optode array.

18. A near-infrared imaging system according to claim 17, wherein each of the plurality of near-infrared radiation detector units comprises an imaging sensor for detecting near-infrared radiation detected by a respective optode that is coupled to the near-infrared radiation detector unit.

19. A near-infrared imaging system according to claim 18, wherein the imaging sensor comprises a plurality of pixels.

20. A near-infrared imaging system according to claim 19, wherein at least one pixel of the imaging sensor is coupled to each optical fibre of the plurality of optical fibres of a respective optode.

21. A near-infrared imaging system according to claim 16, wherein the imaging sensor is a charge coupled device (CCD) or complementary metal oxide semiconductor (CMOS) imaging sensor.

22. A near-infrared imaging system according to claim 16, wherein the target feature is a change in perfusion or liquid content of biological tissue.

23. A near-infrared imaging system according to claim 16, wherein the target feature is indicative of a target pathology.

24. A near-infrared imaging system according to claim 16, wherein the target pathology is an intracranial haematoma, intracranial haemorrhage, or change in blood flow, blood oxygenation or blood volume characteristic of cerebral ischaemia.

25. A near-infrared imaging system according to claim 16, wherein the near-infrared imaging system is a near-infrared spectroscopy or near-infrared tomography system.

26. A near-infrared imaging system according to claim 16, wherein the data processing system is configured to perform a demodulation process on detected near-infrared radiation prior to processing the detected near-infrared radiation in accordance with the near-infrared imaging model.

27. A method of transmitting near-infrared radiation into and/or receiving near-infrared radiation from a subject's head, the method comprising:
bringing a near-infrared imaging optode into contact with a subject's head, the optode comprising a plurality of resilient optical fibres arranged to transmit and/or receive corresponding near-infrared radiation, the plurality of optical fibres each comprising a distal end arranged to make contact with the subject's head, wherein the distal ends of the optical fibres are movable relative to each other, and wherein the optode is arranged to be coupled with a near-infrared radiation detector unit which comprises an imaging sensor; and
transmitting and/or receiving near-infrared radiation via one or more optical fibres of the plurality of optical fibres that are in contact with the subject's head.

28. A method as claimed in claim 27, further comprising: removing the near-infrared imaging optode from contact with the subject's head such that the plurality of optical fibres return to an initial shape.
